(19) United States
(12) Patent Application Publication
(10) Pub. No.: US 2006/0064016 A1
(43) Pub. Date: Mar. 23, 2006

(54) Method and apparatus for automatic examination of cardiovascular functionality indexes by echographic imaging

(75) Inventors: Marcello Demi, Montescudaio (IT); Francesco Faita, La Spezia (IT); Vincenzo Gemignani, Torre del Lago Puccini (IT); Massimo Giannoni, San Ginliano Terme (IT); Antonio Benassi, S. Giuliano Terme-Ghezzano (IT)

Correspondence Address: DENNISON, SCHULTZ, DOUGHERTY & MACDONALD, 1727 KING STREET, SUITE 105, ALEXANDRIA, VA, 22314, US

(73) Assignee: CNR Centro Nazionale delle Ricerche & ESAOTE SpA

(21) Appl. No.: 11/230,825

(22) Filed: Sep. 21, 2005

(30) Foreign Application Priority Data
Sep. 21, 2004 (IT) IT PI2004A000066

(51) Int. Cl. A61B 8/02

(52) U.S. Cl. 600/450

(57) ABSTRACT

A method for automatic examination of echographic images of the cardiovascular system, in particular, for computing functionality indexes of a segment of the cardiovascular system. Said method computes a succession of starting images (1), extracts a succession of images (4) synchronized with a cyclical movement (2) of the examined segment and measures cardiovascular functionality indexes (5) in the succession of synchronized images (4). The method, furthermore, describes the process for defining the edges of the examined structure starting from a starting ultrasonographic image in which the geometric features of the cardiovascular structure are known.

### FIELD OF THE INVENTION

The present invention relates to an automatic method for determining cardiovascular functionality indexes by analyzing sequences of echographic images. In particular, but not exclusively, such images can be obtained from a patient after a treatment of mechanical or pharmacological type, as well as after a treatment that can enhance the capacity of reaction of an object under analysis and then its functionality.

### BACKGROUND OF THE INVENTION

Ultrasonic examination of an organ's function is clinical practice that has quickly spread for its advantages of low invasivity, simplicity, and feasibility. However, especially in cardiovascular analysis, a typical ultrasonic examination by means of manual evaluation of echographic images has relevant practical limits. A typical ultrasonic examination, in fact, for being acceptable in the clinical routine, provides normally a few insulated measurements and introduces a considerable variability. For a more robust examination, for reducing the analysis duration and for reducing the subjectivity of the examination an automatic image analysis is required.

Currently, systems exist capable of analyzing automatically a whole set of successive echographic images and of providing a more objective and robust examination of the functionality of an organ. Such systems allow detecting and analyzing all the images of an echographic succession, or allow analyzing only an image for each cardiac cycle and in the latter case normally a synchronized acquisition with the electrocardiographic signal is involved.

Typically, image acquisition is synchronized with the peak value of the R wave of the electrocardiographic signal, but the R wave is not always in phase with the watched phenomena. For example, if the maximum diameter of a vessel is monitored with time it should be considered that at a desired point of the patient's body the peripheral R wave cannot be in phase with a central R wave. Furthermore, the phase shift of the two events depends on many factors and normally is unsteady with time. Indeed, for example, the phase shift of the peak value of the R wave with respect to the maximum diameter of an artery is unsteady during a transient phase caused by an occlusion of the vessel or when supplying a drug. So, it is not sufficient to add a delay time in the process for acquisition to synchronize the measurement of the maximum diameter of the vessel with the peak value of the R wave.

Another relevant aspect is that an organ can be monitored automatically on sequences of echographic images that are even 30 minutes long, and suitable mathematical operators are necessary for it. The mathematical operators used for controlling the images normally must recognize and localize at high rate the edges of an organ on sequences of images. Many mathematical operators have been studied for improving the performances of algorithms of contour recognition and localization, and the most used in literature remain the Laplacian of Gaussian (LoG) and the Gradient of Gaussian (GoG). Another mathematical operator that provides interesting results and has some advantages is the barycentre of the variability of the grey levels derived by the generalization of the central absolute moment. Other useful mathematical operators can be the average or the median and the variance or the average deviation for calculating respectively the central value and the distribution of the grey levels contained in a region.

In ultrasonic images obtained in a B-mode, some parts of the edge of a structure cannot result enough defined owing to the reverberation of the ultrasonic beam after crossing many layers of tissue. For example, the upper edge of a longitudinal cross section of a vessel is displayed less clearly than the lower edge. In U.S. Pat. No. 6,475,149 the problem has been faced using two different techniques for determining the upper and lower edges: the Doppler signal analysis and the B-mode image analysis, respectively.

Ultrasonic images are also frequently used to obtain the so-called functionality indexes, which are calculated on the basis of measurements, such as thicknesses, diameters, volumes, pressures, etc. Such measurements can be carried out at a determined phase of the cardiac cycle (for example diastolic phase or systolic phase) and can be carried out in coincidence with external, pharmacological, mechanical, thermal actions, etc.

Some functionality indexes are, for example:
ejection fraction;
perfusion of the myocardium;
flow-mediated vessel dilation;
kinetics of the left ventricle;
variability of the cardiac frequency.

### SUMMARY OF THE INVENTION

It is a feature of the present invention to provide a method for carrying out functional cardiovascular measurements synchronized with a cyclical motion of a structure, in particular, with a local kinetics induced by the heart beat, starting from sequences of echographic images and without using further biological signals.

It is a further feature of the present invention to provide a method for defining the edges of a cardiovascular structure, in particular for calculating the diameter of a blood vessel for each image of a succession of images.

It is a particular feature of the present invention to provide a method for calculating the diameter of a blood vessel from sequences of echographic images not synchronized with a particular phase of the cardiac cycle.

It is still a feature of the present invention to provide a method for giving to a medical operator data relating to the correct arrangement of an echographic probe.

These and other objects are achieved, according to an aspect of the invention, by a method for automatic examination of echographic images of the cardiovascular system, in particular, for computing functionality indexes of a segment of the cardiovascular system, comprising the steps of:
obtaining a succession of echographic images of said segment, said images being detected at constant intervals in a predetermined period, in order to provide a local sampling of the cardiovascular activity in said segment;
creating at least one function of time, so-called “reference signal”, measuring at least one scalar or vectorial quantity on all the images of said succession and reporting its course with time;
using this signal for computing said functionality indexes.

In particular, said method provides the further steps of:
choosing at least one conspicuous point of the morphology of said or each reference signal and obtaining a local synchronism signal at each repetition of the conspicuous point in the signal;
   sub-sampling said succession of echographic images by said local synchronism signal;
   providing an output of only the images relative to said sub-sampling, for measuring the cardiovascular functionality in said segment only at the instants defined by said synchronism signal.

This way, the present invention has the advantage to provide a synchronized succession of functional cardiovascular measurements, displaying at the same time the not synchronized images and the reference signal.

Said reference signal can be calculated with a not high precision, but enough for measuring a phase of the local kinetics induced by the heart beat.

In a particular exemplary embodiment, said conspicuous point of the morphology of the reference signals is selected from the group comprised of: a local maximum, a local minimum, an inflexion point or other conspicuous points detectable by applying mathematical operators such as, for example, the first derivative and the second derivative.

Advantageously, said reference signal is a signal of comparison for determining the correct orientation of an echographic probe at the end of the step of determining said functionality indexes.

Always advantageously, said method for automatic examination provides a step of creating a graph of the measured cardiovascular functionality indexes in the presence of transient situations provided by external mechanical, pharmacological, thermal stimuli, etc.

Preferably, the step of measuring said cardiovascular functionality index is selected from the group comprised of:
measuring the diameter of a vessel;
measuring the area of a cross section of a cardiovascular structure;
measuring the average of grey levels or of color levels calculated in a reference window, or in any case a desired scalar or vectorial quantity determinable starting from the images of the succession defined by said synchronism signal.

Advantageously, said cardiovascular functionality indexes are calculated on all the input images of the succession and then sub-sampled by said local synchronism signal.

In a particular exemplary embodiment, said measurements of the cardiovascular functionality indexes are displayed with the succession of the starting images.

Alternatively, said measurements of the cardiovascular functionality indexes are displayed with the reference signal and the synchronism signal.

According to another aspect of the present invention, a method for automatic examination of the cardiovascular functionality by echographic imaging is characterized in that it comprises the steps of:
prearranging a first image where a first edge, so-called “edge of reference” and a second edge, so-called “remaining part of the edge”, of a cardiovascular structure, are defined;
applying on a second image successive to the first an estimated edge in a position exactly corresponding to said edge of reference of the first image;
   and wherein on said second image the following steps are carried out:
creating a first approximated edge distant a fixed amount from said estimated edge;
computing an edge of reference by an “edge detection” algorithm using points belonging to said approximated edge;
from said edge of reference, computing an estimated remaining part of the edge using data on the geometry of the examined structure calculated on images previous to said image;
computing a second approximated edge distant a fixed amount from said estimated remaining part of the edge;
computing a remaining part of the edge by an “edge detection” algorithm using points belonging to said second approximated edge.

In particular, said positive amount is chosen so that said first and second approximated edge are a set of points in said cardiovascular structure.

Advantageously, said edge of reference is the lower edge of a longitudinal cross section of a vessel, i.e. the edge where the echo signal transmitted by the probe encounters firstly the blood and then the tissue.

In particular, said operator of edge detection is a desired mathematical operator that can be used for determining an edge starting from a point near this edge. In particular, it is selected from the group comprised of: the operator of Canny, the Laplacian of Gaussian, the barycentre of dispersion.

Preferably, the echographic probe is located so that said edge of reference is as far as possible orthogonal to the direction of propagation of the ultrasonic signal.

According to another aspect of the present invention, a method for automatic examination of the cardiovascular functionality by echographic imaging is obtained from an apparatus for measuring cardiovascular functionality indexes on sequences of ultrasonic pulse images, comprising:
a first processor that computes all the images of the succession and generates reference signals and a local synchronism signal;
a sub-sampler of images that extracts from the succession of ultrasonic images the images that correspond to synchronism instants;
a second processor that computes the sub-sampled images and extracts the measurements of the cardiovascular functionality indexes.

Advantageously, said apparatus for measuring cardiovascular functionality indexes provides a delay between the succession of input images provided to said first processor and the succession of input images provided to said sub-sampler.

It is relevant, then, according to the invention, to generate a local reference signal from which the sought synchronism signal is obtained. In the present invention, said signal is generated directly by analyzing the same succession of images used for analysis of the organ, thus avoiding to use other instruments and to increase the complexity of the system. This feature characterizes the present invention from other prior art methods that use physiological signals obtained directly from the electrocardiogram for synchronizing the generation of echographic images or a measurement carried out on them.

The present invention is focused preferably on the B-mode analysis of images, by dividing the edge into two parts: the first part of the edge, which is normally clearer, is calculated with a contour-tracking technique, whereas the remaining part of the edge is calculated starting from the first part, by exploiting data that are known a priori from the geometry of the structure.

### BRIEF DESCRIPTION OF THE DRAWINGS

The invention will be made clearer with the following description of an exemplary embodiment thereof, exemplifying but not limitative, with reference to the attached drawings wherein:

FIG. 1 shows, on diagrams, exemplary steps that embody the present invention;

FIGS. 2 and 3 show a correct arrangement of an echographic probe with respect to a blood vessel;

FIG. 4 shows a typical signal that is detected when an echographic probe is located correctly with respect to the vessel;

FIG. 5 shows the variation of a diameter of a vessel with time;

FIG. 6 shows an edge of reference and a remaining part of the edge of reference traced on a representation of a vessel derived by a starting echographic image;

FIGS. 7 to 10 show, on a representation of a vessel derived by an echographic image successive to the starting image, a succession of steps of the present method that eventually define the edge of the blood vessel on said successive image;

FIG. 11 shows actual edges of the cardiovascular structure, obtained with the present method on said successive image.

FIGS. 12 to 15 show respectively four different exemplary embodiments of an apparatus that carry out the present method.

### DESCRIPTION OF PREFERRED EXEMPLARY EMBODIMENTS

The present invention comprises an apparatus that computes a succession of starting images of an examined structure, obtaining a succession of images that are synchronized with respect to a cyclical movement of the structure and measures cardiovascular functionality indexes on this new synchronized succession of images.

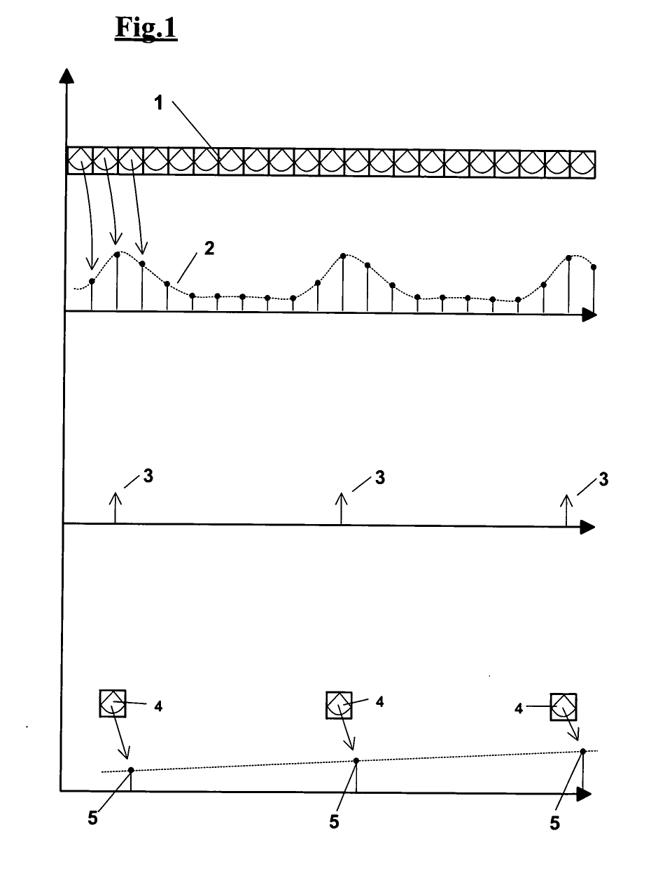

FIG. 1 shows a possible embodiment of the various steps that carry out the present invention. The starting succession 1 is a set of B-mode images or color-Doppler images obtained with a high frame-rate, for example 25 or 30 frame/second. On each image of the succession at least one measure of a quantity is carried out obtaining a time-discrete signal 2, so-called “reference signal”, shown in the figure on a diagram that reports the time in abscissas and the measured quantity in ordinates. Said measure is chosen in order to determine a local kinetics induced by the heart beat. For example the reference signal 2 can be alternatively an area of the structure, a linear quantity, an intensity of the grey levels or the color intensity calculated on a reference window, as well as coordinates of a part of the structure determined by tracking techniques, or in any case a scalar or vectorial quantity determinable starting from the images of the succession. The reference signal 2 is then analyzed by searching conspicuous points corresponding to a same phase of the cardiac cycle. In the example of FIG. 1, said conspicuous points are a local maximum and/or a maximum point in the period of the function. Other criteria for identification of such conspicuous points can be, for example, the search of the minimum of the function or the point of maximum slope.

The instants corresponding to said conspicuous points provide a synchronism signal 3 used for sub-sampling the starting succession 1, obtaining in this way a succession of synchronized images 4, which are also shown in the figure versus time, which is given in the abscissas. The step of sub-sampling consists of the extraction of images 4 from succession 1 exactly at the instants corresponding to the conspicuous points.

Finally, cardiovascular functionality indexes 5 are calculated on each image of the synchronized succession 4. Such indexes can be, for example, an area, a linear quantity, a signal of perfusion or in any case a desired clinical quantity determinable starting from the images. The functionality indexes 5, given in the figure responsive to time, allow to evaluate the functionality of the cardiovascular system.

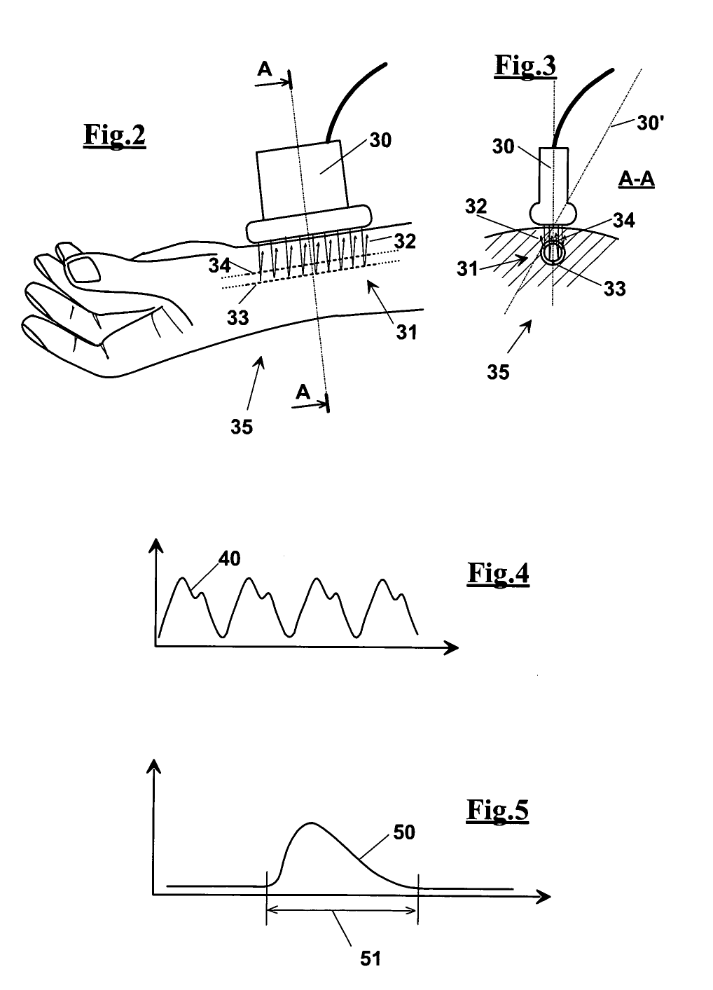

FIGS. 2 and 3 show a correct arrangement of an echographic probe 30 with respect to a patient's 35 blood vessel 31. In particular, FIG. 2 and FIG. 3 show how the direction of propagation of the ultrasonic beam of said echographic probe 30 crosses orthogonally the axis of the blood vessel. The present invention allows positioning correctly said echographic probe 30 with respect to the vessel 31; it is carried out easily since a correct location is achieved when the apparatus that embodies the present method outputs a typical cardiac signal, like the signal 40 shown in FIG. 4, which represents the course with time of a local parameter, for example the diameter of a blood vessel, similar to the signal 2 of FIG. 1.

A disturbed signal would be an evidence of incorrect location, for example an arrangement not parallel to the vessel or inclined according to a line 30′ (FIG. 3).

In the way described hereinafter, the method allows to determine a lower edge 33 of the vessel 31, considered as “edge of reference”, i.e. where the signal 32 transmitted by the echographic probe 30 first encounters the blood and then encounters the tissue, finding a very clear edge, whereas the upper edge 34 will be considered a %%remaining part of the edge”.

In FIG. 5, instead, a diagram is shown that represents the course of a transient 50 of a quantity determined with the present method responsive to time, corresponding to the signal 5 of FIG. 1. The present method allows to determine the starting and end points of the transient same and then its duration 51, thus allowing to limit the examination to its actual range, eventually obtaining a measurement of the functionality indexes which are detected at the conspicuous points, and thus very reliable.

The figures from 6 to 11 describe an application of the method, and in particular the steps of defining the edges of a cardiovascular structure.

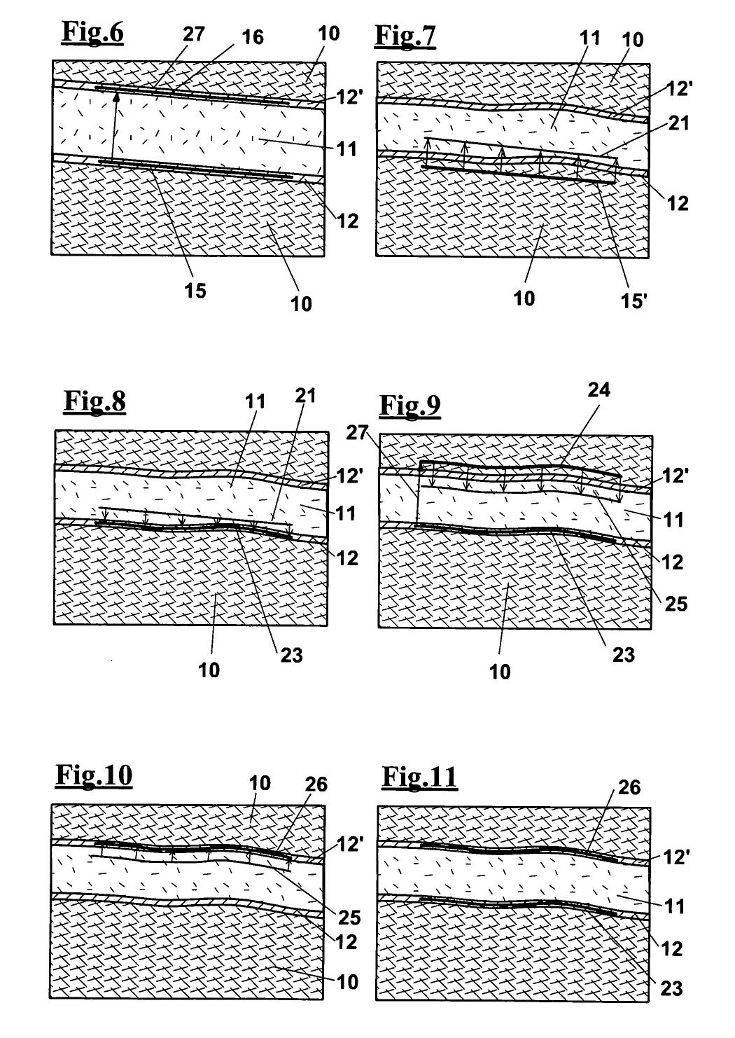

The process for defining the edges begins from a starting ultrasonographic image which is shown in FIG. 6. On said image the geometric features of the cardiovascular structure are already known, for example the edge of reference, the remaining part of the edge of reference and the distance between them.

Figures from 7 to 11 show the successive steps of the process for defining the edge on a representation of a second ultrasonographic image next to the starting image of FIG. 6.

In FIG. 7, an estimation 15′ of the edge of reference is given on the representation of said second ultrasonographic image in a position and shape corresponding to the edge of reference 15 of the starting image of FIG. 6. In the case examined, the estimation of the edge 15′ does not coincide necessarily with the real lower edge 12, since in the second ultrasonographic image the position and the geometry of the vessel are presumably changed with respect to the starting image of FIG. 6. From the estimation 15′ of the edge of reference N points are then obtained in the examined structure which are distant, from said estimated edge of reference, of a distance higher than zero, indicated in FIG. 7 with an arrow. Such points must be enough to provide the approximated edge 21.

In FIG. 8, starting from the approximated edge 21 of FIG. 7, the edge of reference 23 is obtained, by using for approximated edge 21 an edge detection algorithm. This edge of reference 23 represents the position and the actual geometry of the lower edge of the cardiovascular structure examined in the images from FIGS. 7 to 11.

In FIGS. 9 and 10 the definition the remaining part of the edge is effected.

In particular, in FIG. 9 an estimation 24 is obtained of the remaining part of the edge by translating the edge of reference 23, obtained in FIG. 8, for an amount 27 which is equal to the distance between the edge of reference 15 and the remaining part of the edge 16 of the starting image of FIG. 6. From this estimation of edge 24, an approximated edge 25 is obtained, consisting of N points in the cardiovascular structure which are distant more than zero, as indicated by the arrow of FIG. 9.

In FIG. 10, starting from the approximated edge 25, as determined in FIG. 9, the remaining part of the edge 26 is obtained, by using an algorithm of edge detection at the approximated edge 25. Thus, as shown in FIG. 11, the edge of reference 23 and the remaining part of the edge 26, corresponding to the actual geometry and position of the lower edge 12 and upper edge 12′ of the cardiovascular structure, are obtained.

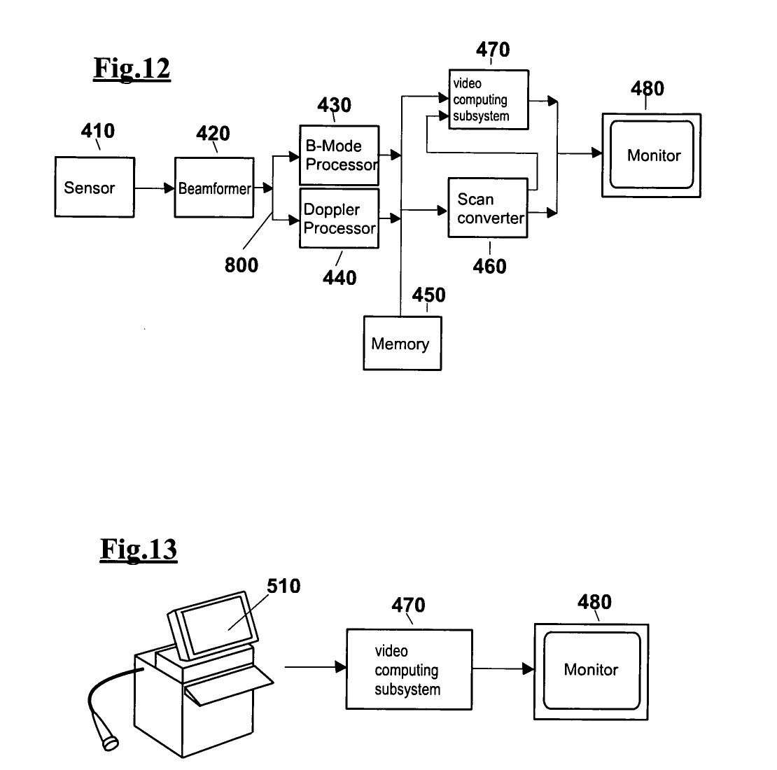

In a preferred exemplary embodiment, shown in FIG. 12, the control of the images is integrated in an echographic apparatus, which comprises a transducer of ultrasonic pulses 410 that transmits the signal to a beam former 420, which supplies a signal 800 to a processor for synthesizing the echographic image 430 and to a processor for exploiting the Doppler signal 440. From the signals at the output of the latter, by a memory 450, a scan-converter 460 and a video control system 470, an image shown by a monitor 480 is obtained.

While in FIG. 12 the image that reaches the monitor 480 has been already processed with the method according to the invention, in a second exemplary embodiment, shown in FIG. 13, it is possible that the images are computed outside form a traditional echographic apparatus 510. In this case an external sub-system is provided with a video control 520 that computes the images generated in a known way by the echograph 510, transforming them according to the described method and displaying them on a further external monitor 530.

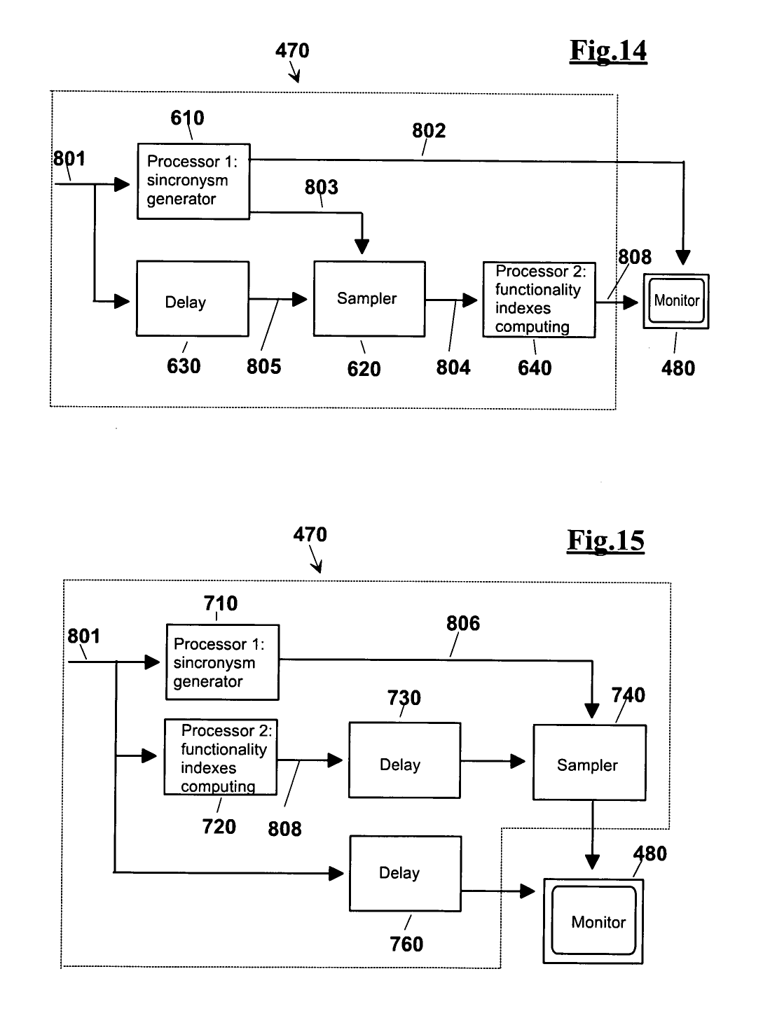

FIG. 14 shows a block diagram of the video control subsystem (470 of FIGS. 12 or 13), comprising the following components:
a first processor 610 that computes all the images of the succession 801 coming from the echograph, generating a reference signal 802, which corresponds to the cyclical movement of the examined structure, and the synchronism signal 803;
a sub-sampler 620 of images that extracts from the succession of images 801 only the images 804 defined by the synchronism pulses 803;
a memory 630 capable of delaying the video succession of a number of images higher than or equal to zero, obtaining a delayed succession 805;
a second processor 640 that computes the sub-sampled images 804 and extracts the measurements of the cardiovascular functionality indexes 808;
the monitor 480 that displays the succession of images, the reference signal 802 and the cardiovascular functionality indexes 808.

In another exemplary embodiment of the sub-system 470, shown in FIG. 15, the cardiovascular functionality indexes are calculated on all the images of the succession and then sub-sampled by the synchronism signal. Also in this case, a first processor 710 computes all the images of the starting succession 801 and generates a reference signal and the relative synchronism signal 806. The calculus of the functionality indexes is also carried out on all the images 801 by a second processor 720 or, alternatively, by the same first processor 710. The not synchronized functionality indexes 808 pass then at a delay unit 730 for being then synchronized by a sampler 740. The synchronized data are then shown on a monitor 480 with the original images, suitably delayed by a unit 760.

The foregoing description of a specific embodiment will so fully reveal the invention according to the conceptual point of view, so that others, by applying current knowledge, will be able to modify and/or adapt for various applications such an embodiment without further research and without parting from the invention, and it is therefore to be understood that such adaptations and modifications will have to be considered as equivalent to the specific embodiment. The means and the materials to realize the different functions described herein could have a different nature without, for this reason, departing from the field of the invention. It is to be understood that the phraseology or terminology employed herein is for the purpose of description and not of limitation.

1. A method for automatic examination of echographic images of a cardiovascular system, in particular, for computing functionality indexes of a segment of the cardiovascular system, comprising the steps of:
obtaining a succession of echographic images of said segment, said images being detected at constant intervals in a predetermined period, in order to provide a local sampling of a cardiovascular activity in said segment;
creating at least one function of time, so-called “reference signal”, by measuring at least one scalar quantity on all the images of said succession and reporting a course of said quantity versus time;
using this signal for computing said functionality indexes.

2. A method, according to claim 1, wherein the further steps are provided of:
choosing at least one conspicuous point of the morphology of said or each reference signal and obtaining a local synchronism signal at each repetition of the conspicuous point of the signal;
sub-sampling said succession of echographic images by said local synchronism signal;
providing an output of only the images relative to said sub-sampling, for measuring cardiovascular functionality indexes in said segment only at instants defined by said synchronism signal.

3. Method, according to claim 1, wherein said reference signal is a signal of comparison for determining a correct orientation of an echographic probe at the end of said step of determining said functionality indexes.

4. Method, according to claim 1, wherein a step is provided of creating a graph of measured cardiovascular functionality indexes in the presence of transient situations provided by external mechanical, pharmacological or thermal stimuli.

5. Method, according to claim 1, characterized in that said scalar quantity is obtained with edge detection techniques and/or contour tracking techniques, starting from echographic images of a type selected from the group comprised of: B-mode and color-Doppler.

6. Method, according to claim 1, characterized in that said scalar quantity is selected from the group comprised of:
measuring a diameter of a vessel;
measuring an area of a cross section of a cardiovascular structure examined;
an average of the grey levels or of color levels calculated on a reference window, or in any case a desired scalar or vectorial quantity determinable starting from the images of the succession.

7. Method, according to claim 2, characterized in that said conspicuous points of the morphology of the reference signals are selected from the group comprised of: local maximum, local minimum, flexion points or other desired conspicuous point detectable by applying mathematical operators, among which the first derivative and the second derivative.

8. Method, according to claim 1, characterized in that the step of measuring said cardiovascular functionality index is selected from the group comprised of:
measuring the diameter of a vessel;
measuring the area of a cross section of a cardiovascular structure examined;
an average of the grey levels or of color levels calculated on a reference window, or in any case a desired scalar quantity determinable starting from the images of the succession defined by said synchronism signal.

9. Method, according to claim 1, wherein said cardiovascular functionality indexes are calculated on all the input images of the succession and then sub-sampled by said local synchronism signal.

10. Method, according to claim 1, wherein said measurements of the cardiovascular functionality indexes are displayed with the succession of the starting images.

11. Method, according to claim 2, wherein said measurements of the cardiovascular functionality indexes are displayed with the reference signal and the synchronism signal.

12. Method for automatic examination of a cardiovascular functionality by echographic imaging, characterized in that it comprises the steps of:
prearranging a first image where a first edge has been defined, so-called “edge of reference” and a second edge, so-called “remaining part of the edge”, of a cardiovascular structure;
applying on a second image successive to the first an estimated edge in a position exactly corresponding to said edge of reference of the first image;
and wherein on said second image the following steps are carried out:
creating a first approximated edge which is distant a fixed amount from said estimated edge;
computing an edge of reference by an “edge detection” algorithm using points belonging to said first approximated edge;
from said edge of reference, computing a remaining part of the edge estimated using data on the geometry of the examined calculated on images previous to said image;
computing a second approximated edge distant a fixed amount from said estimated remaining part of the edge;
computing a remaining part of the edge estimated by an “edge detection” algorithm using points belonging to said second approximated edge.

13. Method, according to claim 12, characterized in that said positive amount is chosen so that said first and second approximated edge are each a set of points in said cardiovascular structure.

14. Method, according to claim 12, characterized in that said edge of reference is a lower edge of a longitudinal cross section of a vessel, i.e. the edge where an echo signal transmitted by a probe encounters first blood and then tissue.

15. Method, according to claim 12, wherein said edge detection operator is a mathematical operator suitable for determining an edge starting from a point near this edge, selected from the group comprised of: the operator of Canny, the Laplacian of Gaussian, the barycentre of dispersion.

16. Method, according to claim 12, whereby the echographic probe is located so that said edge of reference is substantially orthogonal to the direction of propagation of the ultrasonic signal.

17. Apparatus for measuring cardiovascular functionality indexes on sequences of ultrasonic pulse images comprising:
a first processor that computes all the images of the succession and generates reference signals and a local synchronism signal;
a sub-sampler of images that extracts from the succession of ultrasonic images that correspond to synchronism instants;
a second processor that computes sub-sampled images and extracts measurements of cardiovascular functionality indexes.

18. Apparatus, according to claim 17, comprising a delay between the succession of input images provided to the first processor and the succession of input images provided to the sub-sampler.
